(19) United States
(12) Patent Application Publication
(10) Pub. No.: US 2013/0292294 A1
(43) Pub. Date: Nov. 7, 2013

(54) ALLERGY EMERGENCY KIT

(71) Applicant: Brandon J. Wilson, Mokena, IL (US)

(72) Inventor: Brandon J. Wilson, Mokena, IL (US)

(21) Appl. No.: 13/886,856

(22) Filed: May 3, 2013

Related U.S. Application Data
(60) Provisional application No. 61/642,444, filed on May 3, 2012.

(51) Int. Cl. A61F 17/00

(52) U.S. Cl. CPC A61F 17/00
USPC 206/571

(57) ABSTRACT

An allergy emergency kit (AEK) for use in treating urgent allergic reactions in common areas for allergens to exist. Generally speaking, the kit comprises a container for securing allergy medicines, a customizable panel for fastening allergy medicine in an orderly fashion, at least anaphylaxis device fastened to the panel and positioned within the container, and an instruction sheet positioned within the container and listing urgent care details for victims of allergens. Preferably, the anaphylaxis device includes at least one of either an adult-dosed and a child-dosed anaphylaxis device such as an EpiPen or similar epinephrine injection device. The allergy emergency kit may also contain auxiliary allergy medicine, such as diphenhydramine hydrochloride, antihistamine, anti-inflammatory, anti-itch, pain relief, mild sedative, asthma inhaler, ipecac, anti-nausea medicines and other such medicines, herbs or remedies which might be useful in treating the symptoms of an allergic reaction.

### RELATED APPLICATIONS

The present application claims the filing priority of U.S. Provisional Application No. 61/642,244 titled “Emergency Allergy Kit” and filed on May 3, 2012. The '244 application is hereby incorporated by reference.

### FIELD OF THE INVENTION

The present device relates to an emergency kit. Particularly, the present device relates to a readily accessible and conspicuous kit for urgent care of child and adult allergic reactions.

### BACKGROUND OF THE INVENTION

Some studies have estimated that as much as five percent of the population may have some form of a food allergy. Another 1-5% of the population may suffer anaphylaxis as a result of a bee (or similar insect) sting. Estimates also show that about 40 people die in the United States alone every year from a venom allergy. Even where the allergic reaction is less than deadly, it can be quite serious, resulting in shortness of breath, swelling, severe pain, nausea, vomiting, cramping or similar symptoms.

Further, as with many food allergies, a great number of people are unaware of their allergies until they have a first critical episode i.e., until they eat a specific food or get stung. That means, many of these people, which includes a great many children, do not have the knowledge to be carrying proper allergy medicine.

The present invention solves these problems by providing a kit which can be placed in school, including school cafeterias, seafood-serving, restaurants, other public and private outdoor music venues, public restrooms, sports fields, golf courses, parks, along hiking trails, picnic areas, and wherever people are expected to gather to eat or encounter venomous insects.

### SUMMARY OF THE INVENTION

A new and useful allergy emergency kit (AEK) is described and claimed. Generally speaking, the allergy emergency kit comprises a container having a cover (e.g., a hinged door or panel) for temporarily securing items, such as child-dosed and adult-dosed anaphylaxis devices, and an instruction sheet positioned within the container listing urgent care details for victims of allergens. The container can be a small box-like container for holding a few pre-selected allergy medicines or devices, or it may be a larger cabinet-like container for holding specific allergy items for an entire school or classroom, for example.

In a specific embodiment, the at least one adult dosed anaphylaxis device is an EpiPen® or similar epinephrine injection device. Similarly, the at least one child dosed anaphylaxis device is also an EpiPen® or similar epinephrine injection device.

In another embodiment, the cover comprises a breakable safety panel of a material such as plastic or glass. A “hammer” for breaking the safety glass may also be provided and attached to the container. However, the panel may also be a hinged door or panel to allow full or limited access to the container contents.

The emergency allergy kit may also comprise auxiliary allergy medicine positioned within the container. These auxiliary medicines may include diphenhydramine hydrochloride, antihistamine, anti-inflammatory, anti-itch cream, pain reliever, mild sedative, ipecac, asthma inhaler, anti-nausea medicine, herbal remedies which might be useful in treating the symptoms of nausea or an allergic reaction, and any combination of these medicines. The AEK may also include a standard first-aid kit.

In another embodiment of the emergency allergy kit, the container is mountable to a structure, such as a wall, tree, post, or similar structure.

These and other aspects of the various embodiments of the present invention will be more readily understood by a person of skill in the art upon reading the following description in view of the appended drawings.

### BRIEF DESCRIPTION OF THE DRAWINGS

For the purpose of facilitating an understanding of the subject matter sought to be protected, there are illustrated in the accompanying drawings embodiments thereof, from an inspection of which, when considered in connection with the following description, the subject matter sought to be protected, its construction and operation, and many of its advantages should be readily understood and appreciated.

FIG. 1 is a front view of an embodiment of the present invention, whereby allergy medicine and first aid instructions are stored within a bright, conspicuous case with emergency breakable glass;

FIG. 2 is a front view of an embodiment of the present invention illustrating an alternative hinged door for accessing stored medicine and information;

FIG. 3 is front view of a locked cabinet embodiment of the present invention;

FIG. 4 is a perspective view of the embodiment of FIG. 3;

FIG. 5 is a front view of a larger embodiment of the present invention, which is used to store several personal allergy devices (e.g., allergy pens);

FIG. 6 is a perspective view of one embodiment of a customizable panel for fastening medications within the AEK; and

FIG. 7 is front view of an embodiment of a placard used to identify an AEK.

### DETAILED DESCRIPTION OF PREFERRED EMBODIMENTS

While this invention is susceptible of embodiments in many different forms, there is shown in the drawings and will herein be described in detail a preferred embodiment of the invention with the understanding that the present disclosure is to be considered as an exemplification of the principles of the invention and is not intended to limit the broad aspect of the invention to embodiments illustrated.

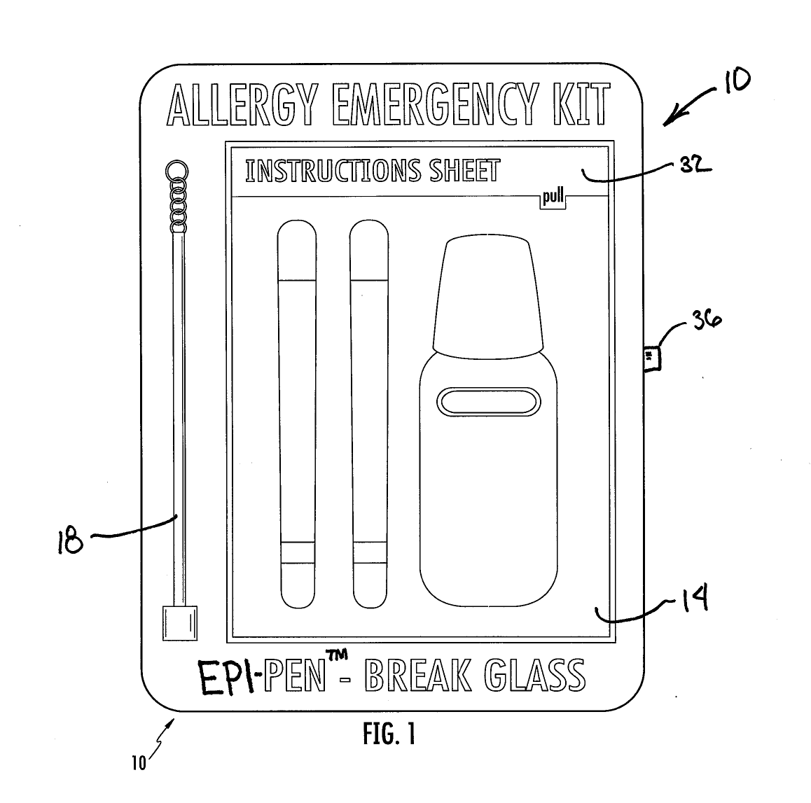

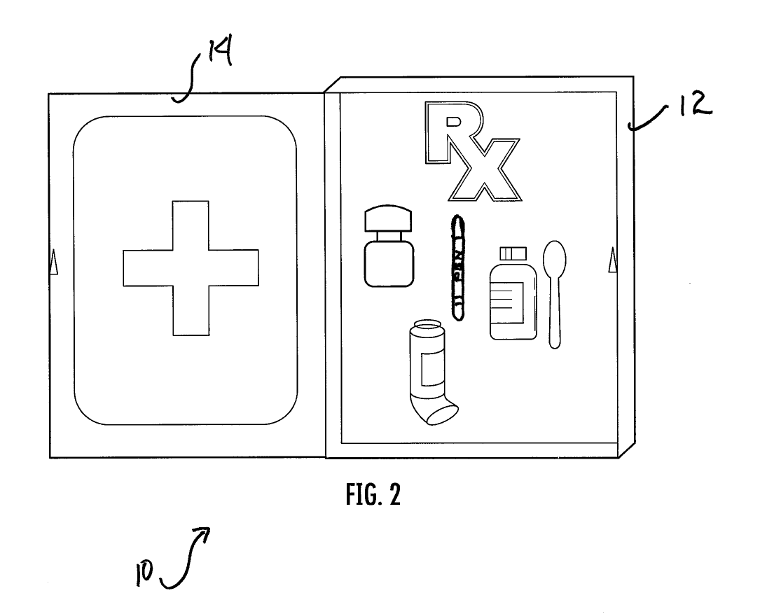

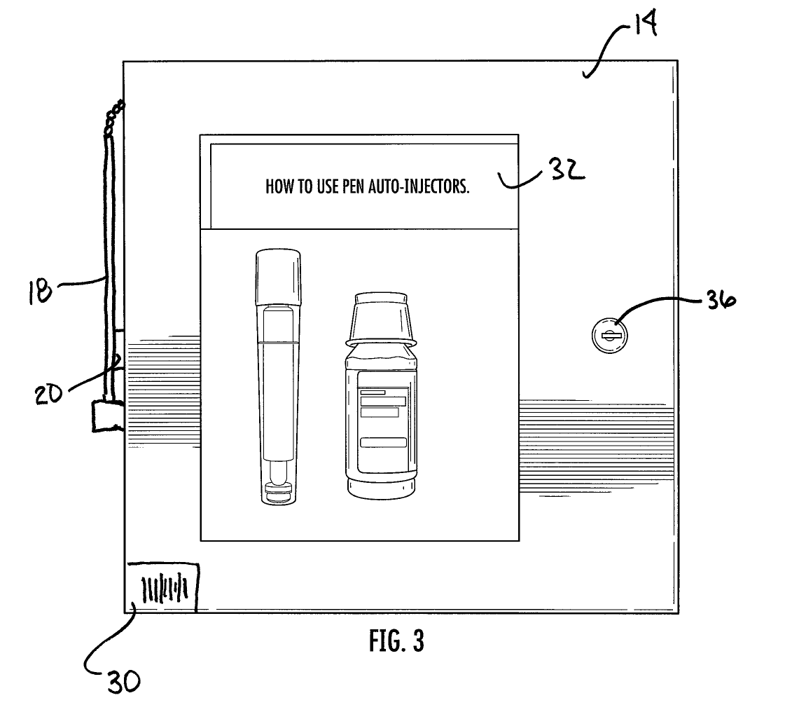

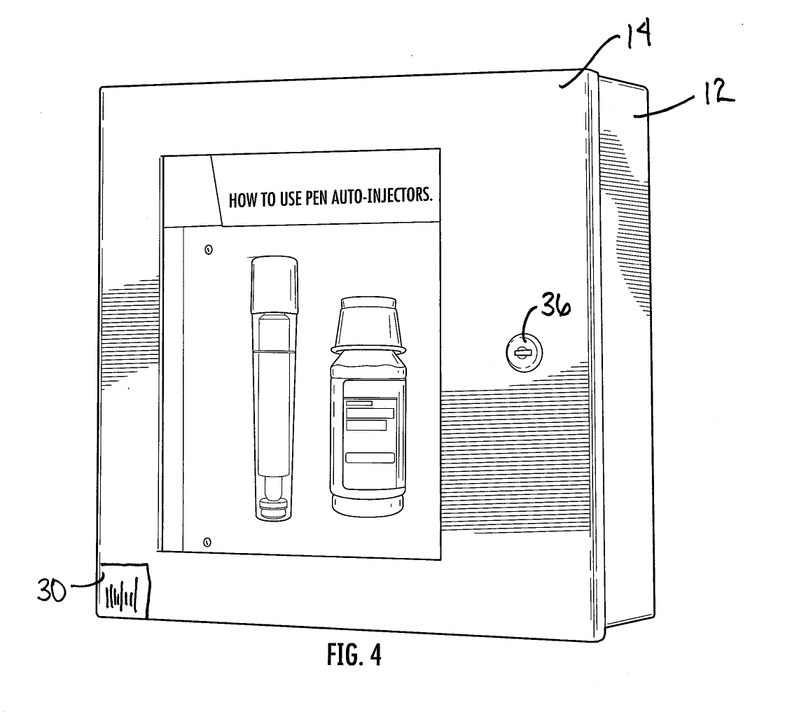

Referring to FIGS. 1-7, there are illustrated embodiments of an Allergy Emergency Kit (AEK), generally designated by the numeral 10, and components thereof. A preferred AEK 10 is comprised of a container 12, such as a cabinet, having an interior area accessible via a front panel 14. The panel 14 may opened, or otherwise removed, to provide access to the interior of the container 12. A customizable panel 16 is positioned within the container 12 to help organize items within the container 12. Those items preferably include at least one adult dose of epinephrine, preferably by injection (e.g., an EpiPen®), at least one child dose of epinephrine, also preferably by injection, auxiliary allergy medicine (e.g., BENADRYL® manufactured and distributed by Johnson & Johnson) and allergy first aid instructions. The AEK 10 may also include a sheet of emergency numbers, cold packs, bandages, over-the-counter (OTC) medicines and other similar first aid treatments. In other preferred embodiments, the AEK 10 might include just adult dosing devices, just child dosing devices, or only other allergy medications, as allowed by law.

Such OTC medicines might include other allergy medicines, similar to Benedryl and having diphenhydramine hydrochloride, antihistamine, anti-inflammatory, anti-itch cream, pain relief medicine, mild sedative, ipecac, asthma inhaler, anti-nausea medicines and other such medicines, herbs or remedies which might be useful in treating the symptoms of nausea and/or an allergic reaction.

Given the numerous state and federal statutes, regulations and local practices concerning the distribution of prescription and over-the-counter medication, the AEK 10 may be initially packaged with or without epinephrine or other medicines. In some circumstances, the end user will need to separately obtain the required or desired medications for storage within the container. Further and more specific details of embodiments of the AEK 10 are set forth below.

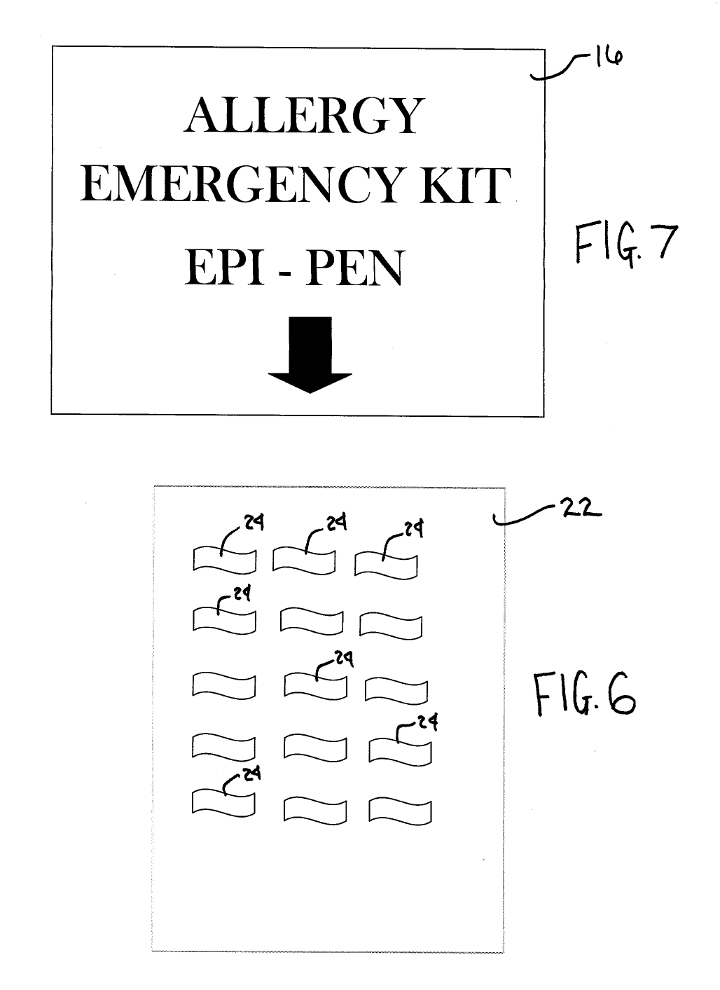

The container 12 of kit 10 may be made from any suitable material, including plastic and metal, and should be designed to be quickly noticed and, for most uses, easily opened. Preferably, the case is white (or substantially white) adorned with red-lettering, or alternately red with white-lettering. While it is not intended to be a first-aid kit, per se, it may be advantageous to include the iconic “red-cross” symbol (or other easily recognized symbol) on the kit 10 to universally designate the box as an aid station. Other symbols, such as insects, poison ivy, peanuts, etc. may also be shown on the box to indicate that the contents are for treatment of such encounters—symbols may be more helpful where persons are unable to read, such as children or non-English speaking persons. The AEK 10 may be designed as a stand-alone container, or for mounting to a wall, post, door, or similar structure. Most importantly, the AEK 10 must be mounted where it can be readily accessed. If desired, a placard 16, an embodiment of which is shown in FIG. 7, may be posted above the AEK 10 to provide additional identification. Similar to the AEK 10 itself, the placard 16 may include insignia to draw attention to the placard.

The AEK 10 includes a cover 14 such as a latched door or a transparent cover plate which is easily removed or even broken (much like emergency alarms in buildings) to provide access to the contents. The use of the breakable cover plate may be preferred to dissuade theft in some areas. To facilitate breaking the glass, a hammer-like device 18, as shown in FIG. 1, may be provided as part of the AEK 10. The hammer-like device 18 may be secured by a chain, for example, and/or it may be held by a magnet 20 to a metal AEK exterior. A lock 36 may be provided on the front or side of the AEK 10 for added security.

For some systems, a battery or power-cell operated signal (visual, audible, electronic or any combination) may be initiated when the kit is breeched (e.g., when the glass is broken). This will provide notice to personnel when the AEK 10 is in need of restocking and/or an emergency is occurring.

In this regard, the present invention provides a practical solution to making the medicine accessible for emergency situations. The container 12 and breakable cover 14 secure the epinephrine (and other allergy medication) until the glass is broken or an alarm is sounded. While some states allow school nurses, for example, to administer such medicine, having the medicine stored in a locked cabinet/drawer possibly in a locked room, makes the accessibility of the life-saving medicine impractical for emergency situations.

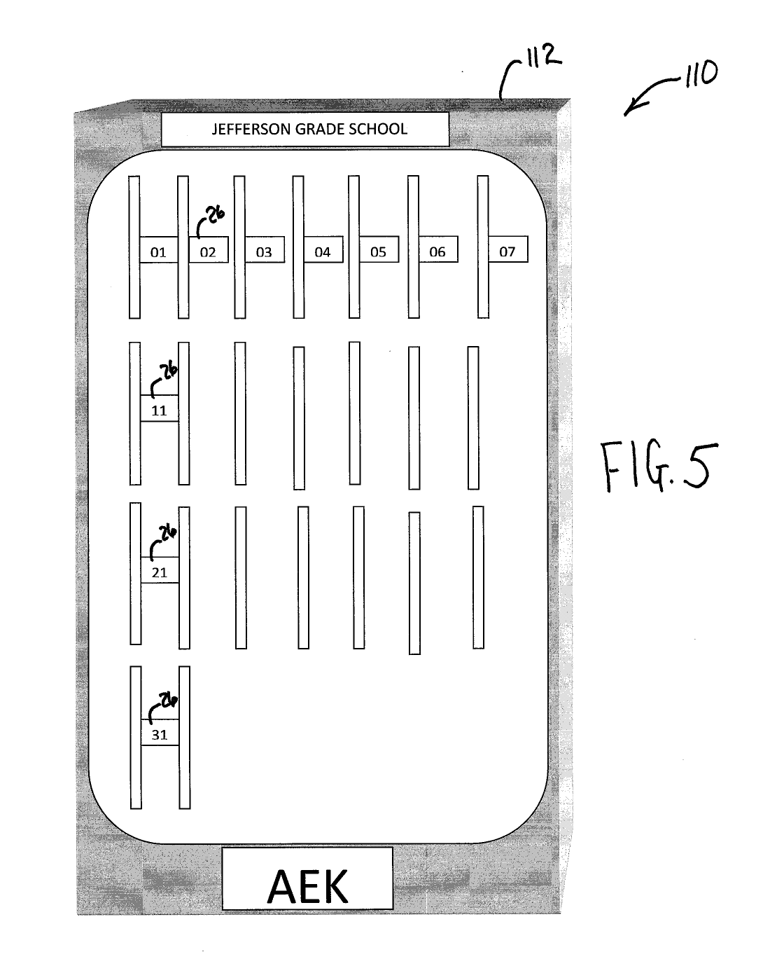

FIG. 5 illustrates a specific embodiment of the AEK 110. A larger cabinet 112 is used to store specific personal medications—e.g., EPI-PENS, inhalers, etc.—for an entire group, such as a classroom, school, business, or the like. A customizable panel 22, see FIG. 6, within the kit includes a plurality of hook-and-loop (i.e., VELCRO) sites 24 for fastening such personal medications. Each site may be labeled 26, for example, with a user's name or ID no. to alleviate accidental misuse. The panel 22 allows multiple medications to be stored in a more organized fashion without the need to sort and hunt through a desk drawer for the proper medicine.

Where prescription medicines are used, such as epinephrine, each AEK 10 may be assigned a specific code 30 for monitoring and tracking the location of medicine. Codes may be simple sequential ID numbers, barcodes, RFIDs, or the like.

Behind the medicine, it is preferred that a laminated emergency instruction sheet 32 or booklet be provided. There are numerous standard first aid measures which can be taken in the event of an allergic reaction and the instructions should list as many of such measures as possible. Also, by laminating the instructions and making them removable from the AEK 10, the instructions may be brought to the allergy victim and maintain structural rigidity, readability and reusability over time. If a booklet is provided, it should be tabbed to allow a user to go immediately to the particular allergy of concern—i.e., food, insect, plant. The front cover of the instructions/booklet may also list general treatment protocol where the specific allergen is unknown.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. An allergy emergency kit comprising:
a container having a removable cover for temporarily securing items therein;
at least one anaphylaxis device positioned within the container; and
an instruction sheet positioned within the container and listing urgent care details for victims of allergens.

2. The allergy emergency kit of claim 1, wherein the at least one anaphylaxis device comprises at least one of either an adult-dosed or a child-dosed EpiPen® or similar epinephrine injection device.

3. The allergy emergency kit of claim 1, wherein the at least one anaphylaxis device comprises at least one adult-dosed and at least one child-dosed EpiPen® or similar epinephrine injection device.

4. The allergy emergency kit of claim 1, wherein the removable cover comprises a breakable safety panel of a material such as plastic or glass.

5. The allergy emergency kit of claim 1, further comprising auxiliary allergy medicine positioned within the container.

6. The allergy emergency kit of claim 5, wherein the auxiliary allergy medicine is selected from the group consisting of diphenhydramine hydrochloride, antihistamine, anti-inflammatory, pain reliever, mild sedative, ipecac, anti-nausea medicine, herbal remedies which might be useful in treating the symptoms of nausea or an allergic reaction, and any combination of these medicines.

7. The allergy emergency kit of claim 1, wherein the container is mountable to a structure.

8. The allergy emergency kit of claim 1, further comprising a first aid kit positioned within the container.

9. The allergy emergency kit of claim 1, further comprising a code on the container to permit management and restocking of the contents.

10. The allergy emergency kit of claim 9, wherein the code is one of either an RFID or a bar code.

11. The allergy emergency kit of claim 1, further comprising a customizable panel within the container for securing a plurality of items.

12. The allergy emergency kit of claim 11, wherein the customizable panel comprises a plurality of hook-and-loop sites for fastening each of the at least one anaphylaxis devices.

13. An allergy emergency kit comprising:
a container having a removable transparent cover for temporarily securing items therein;
a customizable panel including a plurality of hook-and-loop sites for fastening items;
at least one adult-dosed anaphylaxis device fastened to the customizable panel within the container;
at least one child-dosed anaphylaxis device fastened to the customizable panel within the container;
at least one additional first aid item positioned within the container, the at least one additional first aid item being selected from the group consisting of bandages, gauze, burn ointment, anti-bacterial cream, anti-itch cream, anti-fungal cream, anti-inflammatory medicine, asthma inhaler, pain medicine, topical antibiotic cream, over-the-counter medicine, cold-packs, heat-packs, eye-wash, analgesics and anti-venom; and
an instruction sheet positioned within the container and listing urgent care details for victims of allergens.

14. The allergy emergency kit of claim 13, wherein the container is mountable to a structure.

15. The allergy emergency kit of claim 13, further comprising a first aid kit positioned within the container.

16. The allergy emergency kit of claim 13, further comprising a code on the container to permit management and restocking of the contents.

17. The allergy emergency kit of claim 16, wherein the code is one of either an RFID or a bar code.
